(19) United States
(12) Patent Application Publication
(10) Pub. No.: US 2003/0087311 A1
(43) Pub. Date: May 8, 2003

(54) Method of identifying energy transfer sensors for analytes

(76) Inventor: David E. Wolf, Sudbury, MA (US)

Correspondence Address: Allison Johnson, P.A., 6016 Logan Ave. S., Minneapolis, MN, 55419, US

(21) Appl. No.: 10/290,971

(22) Filed: Nov. 7, 2002

Related U.S. Application Data
(60) Provisional application No. 60/337,800, filed on Nov. 7, 2001.

(51) Int. Cl. C12Q 1/68; G01N 33/53; C12M 1/34; G01N 33/543

(52) U.S. Cl. 435/7.1; 436/518; 435/6; 435/287.2

(57) ABSTRACT

A method of identifying an analyte-ligand binding pair that exhibits non-radiative fluorescence resonance energy transfer (FRET) using a combinatorial library. The method includes a) obtaining an analyte binding ligand from a combinatorial library that includes ligands, and b) attaching a label at least one of the analyte binding ligand and an analyte-analogue with at least one of a first component and a second component of a non-radiative fluorescence resonance energy transfer donor-acceptor pair (FRET pair) such that FRET occurs when the analyte-analogue is bound to the analyte binding ligand, and a change in FRET occurs when the analyte-analogue is not bound to the analyte binding ligand.

The method also includes contacting a combinatorial library of ligands, which are optionally labeled with a component of a FRET pair, with analyte-analogue, which is optionally labeled with a component of a FRET pair, and detecting the presence of FRET.

### CROSS-REFERENCE TO RELATED APPLICATIONS

This application claims the benefit of U.S. Provisional Application No. 60/337,800, filed Nov. 7, 2001.

### BACKGROUND

The invention is directed to identifying energy transfer sensors for analytes using a combinatorial library.

Fluorescence provides a highly sensitive mode of analyte detection. Under normal conditions and depending upon background levels, fluorescence can typically detect concentrations as low as nanomolar and picomolar. Additionally fluorescence measurements require small sample volumes less than microliters. As a result fluorescence can typically detect less than 10-18 moles of an analyte within the sample volume. Under more specialized conditions, fluorescence has been used to detect single molecules.

The use of fluorescence in sensor applications has been limited by the need to develop ligands whose fluorescence is specifically sensitive to a given analyte. Examples of this are the FURA family of calcium sensitive fluorescent dyes and the BCECF (i.e., 2′,7′-bis-(2-carboxyethyl)-5(and 6)-carboxyfluorescein) family of pH sensitive fluorescent dyes both of which are described, e.g., in Richard Haugland, “Handbook of Fluorescent Probes and Research Products Ninth Edition Molecular Probes, Eugene, Oreg. 2002, and a fluorescent analogue of UDP-galactose (e.g., 2′(or 3′)-O-(2,4,6-trinitrophenyl)-5′-uridine diphosphate galactose, see, e.g., U.S. Pat. No. 5,109,126), which is sensitive to the enzyme and putative adhesion molecule galactosyl-transferase. In the past, analyte sensitive fluorescent ligands were developed and “tailor-made” on a case by case basis. Often, the resulting chemistries offered little in the way of capability to modulate the affinity of the ligand for analyte, making the effectiveness of such biosensors “catch as catch can.”Additionally, such sensors are often fluorescent at ultraviolet (UV) or near-UV wavelengths, making them of limited use in medical applications.

Combinatorial chemistries have been utilized to create highly specific ligands to a wide variety of potential therapeutic targets. Such libraries include, e.g., mammalian antibody libraries, antibody analogue libraries, apatamer libraries, in vitro peptide libraries, and in vivo peptide libraries created, for example, by phage display.

### SUMMARY

The invention features a method of screening a combinatorial library for a ligand, i.e., the analyte binding ligand, that selectively binds an analyte of interest using an analyte-analogue created from the chemical structure of the analyte. The analyte-analogue is labeled with one element of a non-radiative fluorescence energy transfer (FRET) donor-acceptor pair to create a fluorescent analyte-analogue. The analyte binding ligand is labeled with the conjugate element of the FRET donor-acceptor pair to create a fluorescent analyte binding ligand. When the fluorescent labeled analyte-analogue and the fluorescent labeled analyte binding ligand are mixed, FRET occurs. The presence of analyte is detected by a diminution in the amount or efficiency of FRET.

In a first aspect, the invention features a method of identifying an analyte-ligand binding pair that exhibits non-radiative fluorescence resonance energy transfer, the method including: a) obtaining a predetermined analyte binding ligand from a combinatorial library including ligands, the analyte binding ligand having been predetermined by contacting the combinatorial library with a first analyte-analogue and selecting a ligand to which the first analyte-analogue binds; and b) attaching a label to at least one of the analyte binding ligand and a second analyte-analogue, the label including at least one of a first component and a second component of a non-radiative fluorescence resonance energy transfer donor-acceptor pair such that non-radiative fluorescence resonance energy transfer occurs when the second analyte-analogue is bound to the analyte binding ligand, and a change in non-radiative fluorescence resonance energy transfer occurs when the second analyte-analogue is not bound to the analyte binding ligand. In one embodiment, prior to obtaining the predetermined analyte binding ligand, the predetermined analyte binding ligand includes a label including the first component of the non-radiative fluorescence resonance energy transfer donor acceptor pair. In another embodiment, the method further includes attaching the second component of the non-radiative fluorescence resonance energy transfer donor-acceptor pair to the second analyte-analogue. In other embodiments, the method further includes attaching the first component of the non-radiative fluorescence resonance energy transfer donor-acceptor pair to the analyte binding ligand and attaching the second component of the non-radiative fluorescence resonance energy transfer donor-acceptor pair to the second analyte-analogue.

In one embodiment, the label further includes a linking moiety attached to the analyte binding ligand and at least one of the first component and the second component of the non-radiative fluorescence resonance energy transfer donor-acceptor pair, the moiety being capable of being bound to the analyte binding ligand and at least one of the first component and the second component of the non-radiative fluorescence resonance energy transfer donor-acceptor pair. In other embodiments, the method further includes attaching a linking moiety to at least one of the analyte binding ligand and at least one of the first component and the second component of the non-radiative fluorescence resonance energy transfer donor-acceptor pair, the moiety being capable of being bound to the analyte binding ligand and at least one of the first component and the second component of the non-radiative fluorescence resonance energy transfer donor-acceptor pair.

In other embodiments, the method further includes attaching the first component and the second component of the non-radiative fluorescence resonance energy transfer donor-acceptor pair to the analyte binding ligand. In another embodiment, the method further includes attaching the first component and the second component of the non-radiative fluorescence resonance energy transfer donor-acceptor pair to the second analyte-analogue.

In some embodiments, the combinatorial library further includes a library selected from the group consisting of peptide library, antibody library, antibody fragment library, nucleic acid library, apatamer library, polymer library, and combinations thereof. In another embodiment, the ligands are selected from the group consisting of polymers, antibodies, antibody fragments, nucleotides, peptides, apatamers, and combinations thereof.

In other embodiments, the second analyte-analogue has the same chemical structure as the first analyte-analogue. In some embodiments, the second analyte-analogue has a different chemical structure from the first analyte-analogue.

In a second aspect, the invention features a method of identifying an analyte-ligand binding pair that exhibits non-radiative fluorescence resonance energy transfer, the method including: a) contacting a combinatorial library with an analyte-analogue, the combinatorial library including ligands, b) identifying at least one ligand to which the analyte-analogue binds, the ligand being the analyte binding ligand, and c) attaching a label to at least one of the analyte binding ligand and the analyte-analogue, the label including at least one of a first component and a second component of a non-radiative fluorescence resonance energy transfer donor-acceptor pair such that non-radiative fluorescence resonance energy transfer occurs when the analyte-analogue is bound to the analyte binding ligand, and a change in non-radiative fluorescence resonance energy transfer occurs when the analyte-analogue is not bound to the analyte binding ligand.

In one embodiment, the method further includes attaching a first component of a non-radiative fluorescence resonance energy transfer donor-acceptor pair to the analyte binding ligand. In other embodiments, the method further includes attaching a first component of a non-radiative fluorescence resonance energy transfer donor-acceptor pair to the ligands of the combinatorial library prior to contacting the combinatorial library with the analyte-analogue. In some embodiments, the method further includes attaching a first component of a non-radiative fluorescence resonance energy transfer donor-acceptor pair to the analyte-analogue prior to contacting the combinatorial library with the analyte-analogue. In another embodiment, the method further includes attaching the first component and the second component of the non-radiative fluorescence resonance energy transfer donor-acceptor pair to the analyte binding ligand.

In some embodiments, the method further includes attaching the first component and the second component of the non-radiative fluorescence resonance energy transfer donor-acceptor pair to the ligands of the combinatorial library prior to contacting the combinatorial library with the analyte-analogue. In other embodiments, the method further includes attaching the first component and the second component of the non-radiative fluorescence resonance energy transfer donor-acceptor pair to the analyte-analogue.

In another embodiment, the method further includes attaching the first component and the second component of the non-radiative fluorescence resonance energy transfer donor-acceptor pair to the analyte-analogue prior to contacting the combinatorial library with the analyte-analogue.

In some embodiments, the method further includes selecting an analyte binding ligand to which the analyte-analogue exhibits reversible binding.

In other embodiments, the analyte includes glucose.

In a third aspect, the invention features a method of identifying an analyte-ligand binding pair that exhibits non-radiative fluorescence resonance energy transfer, the method including a) contacting a combinatorial library including a plurality of ligands with an analyte-analogue such that the analyte-analogue binds to at least one of the ligands to form an analyte-ligand binding pair, the ligands including a first label including a first component of a non-radiative fluorescence resonance energy transfer donor-acceptor pair, at least one of the analyte-analogue and the ligands including a second label including a second component of a non-radiative fluorescence resonance energy transfer donor-acceptor pair, and b) detecting an analyte-ligand binding pair that exhibits non-radiative fluorescence resonance energy transfer. In one embodiment, the method further includes identifying the analyte-ligand binding pair. In other embodiments, the identifying and the detecting occur simultaneously or substantially simultaneously. In some embodiments, the method further includes identifying an analyte-analogue-ligand binding pair that exhibits a change in non-radiative fluorescence resonance energy transfer in the presence of analyte.

In other embodiments, at least one of the first and second components of the non-radiative fluorescence resonance energy transfer donor acceptor pair is selected from the family of green fluorescent proteins.

In another embodiment, the method further includes selecting an analyte binding ligand to which the analyte-analogue exhibits reversible binding.

In other embodiments, the detecting is selected from the group consisting of (a) measuring the appearance or disappearance of emission peaks, (b) measuring the ratio of the signal observed at two or more emission wavelengths, (c) measuring the appearance or disappearance of excitation peaks, (d) measuring the ratio of the signal observed at two or more excitation wavelengths and combinations thereof. In some embodiments, the detecting includes measuring the change in the excited state lifetime of the fluorescence. In another embodiment, the detecting includes measuring the depolarization of fluorescence relative to excitation.

In a fourth aspect, the invention features a method of identifying an analyte-ligand binding pair that exhibits non-radiative fluorescence resonance energy transfer, the method including determining a constant region on a ligand at which to attach at least one component of a non-radiative fluorescence resonance energy transfer donor-acceptor pair, b) obtaining a predetermined analyte binding ligand from a combinatorial library including ligands including the predetermined constant region, the analyte binding ligand having been predetermined by contacting the combinatorial library with a first analyte-analogue, and selecting an analyte binding ligand capable of binding the first analyte-analogue, and c) attaching a label including at least one of a first component and a second component of the non-radiative fluorescence resonance energy transfer donor-acceptor pair to at least one of the analyte binding ligand and a second analyte-analogue such that non-radiative fluorescence resonance energy transfer occurs when the second analyte-analogue is bound to the analyte binding ligand, and a change in non-radiative fluorescence resonance energy transfer occurs when the second analyte-analogue is not bound to the analyte binding ligand. In some embodiments, the method further includes attaching a label including the first component of the non-radiative fluorescence resonance energy transfer donor-acceptor pair to the analyte binding ligand at the predetermined constant region on the analyte binding ligand, and attaching a label including the second component of the non-radiative fluorescence resonance energy transfer donor-acceptor pair to at least one of the analyte binding ligand and the second analyte-analogue.

In other embodiments, the method further includes preparing a combinatorial library including ligands including the constant region, contacting the combinatorial library with a first analyte-analogue, and identifying a ligand to which the first analyte-analogue binds, the ligand being the analyte binding ligand. In one embodiment, the preparing includes attaching a label including at least one component of the non-radiative fluorescence resonance energy transfer donor acceptor pair to the constant region of the ligands of the combinatorial library.

In other embodiments, the constant region of the ligands includes at least one component of the non-radiative fluorescence resonance energy transfer donor acceptor pair.

In some embodiments, the second analyte-analogue includes a predetermined constant region capable of binding at least one component of the non-radiative fluorescence resonance energy transfer donor-acceptor pair.

In another embodiment, the method further includes attaching a label including the first component of the non-radiative fluorescence resonance energy transfer donor-acceptor pair to the constant region of the analyte binding ligand, and attaching a label including the second component of the non-radiative fluorescence resonance energy transfer donor-acceptor pair to the constant region of the second analyte-analogue.

In other embodiments, the method further includes selecting an analyte binding ligand to which the second analyte-analogue exhibits reversible binding.

In a fifth aspect, the invention features a method of identifying an analyte-ligand binding pair that exhibits non-radiative fluorescence resonance energy transfer, the method including determining a region on an analyte-analogue at which to attach a component of a non-radiative fluorescence resonance energy transfer donor-acceptor pair, preparing an analyte-analogue including the predetermined region, contacting a combinatorial library including ligands with the analyte-analogue, identifying a ligand to which the analyte-analogue binds, the ligand being the analyte binding ligand, and attaching a label including at least one of a first component and a second component of a non-radiative fluorescence resonance energy transfer donor-acceptor pair to at least one of the analyte binding ligand and the analyte-analogue such that non-radiative fluorescence resonance energy transfer occurs when the analyte-analogue is bound to the analyte binding ligand, and a change in non-radiative fluorescence resonance energy transfer when the analyte-analogue is not bound to the analyte binding ligand. In one embodiment, the method further includes attaching at least one component of the non-radiative fluorescence resonance energy transfer donor acceptor pair to the constant region of the analyte-analogue. In other embodiments, the method further includes selecting an analyte binding ligand to which the analyte-analogue exhibits reversible binding.

In some embodiments, the identifying and the selecting occur simultaneously or substantially simultaneously.

In a sixth aspect, the invention features a method of identifying an analyte-ligand binding pair that exhibits non-radiative fluorescence resonance energy transfer, the method including a) identifying a linking moiety to which at least one component of a non-radiative fluorescence resonance energy transfer donor-acceptor pair binds, b) obtaining a predetermined analyte binding ligand from a combinatorial library including ligands, the analyte binding ligand having been predetermined by contacting the combinatorial library with a first analyte-analogue, and selecting an analyte binding ligand capable of binding the first analyte-analogue, and c) attaching a label to the linking moiety, the label including a first component of a non-radiative fluorescence resonance energy transfer donor-acceptor pair, d) attaching a label including a second component of the non-radiative fluorescence resonance energy transfer donor-acceptor pair to at least one of the analyte binding ligand and a second analyte-analogue, and e) attaching the linking moiety to the analyte binding ligand, wherein non-radiative fluorescence resonance energy transfer occurs when the second analyte-analogue is bound to the analyte binding ligand, and a change in non-radiative fluorescence resonance energy transfer when the second analyte-analogue is not bound to the analyte binding ligand. In some embodiments, the method further includes attaching the label to the linking moiety prior to attaching the moiety to the analyte binding ligand.

In a seventh aspect, the invention features a method of screening a combinatorial library, the method including a) preparing a combinatorial library including ligands including a first component of a non-radiative fluorescence resonance energy transfer donor-acceptor pair, b) contacting the combinatorial library with an analyte-analogue including a second component of a non-radiative fluorescence resonance energy transfer donor-acceptor pair; and c) identifying an analyte-ligand binding pair that exhibits non-radiative fluorescence resonance energy transfer.

In an eighth aspect, the invention features a sensor that includes an analyte-ligand binding pair including a first analyte-analogue and a predetermined analyte binding ligand, the analyte binding ligand having been predetermined by contacting a combinatorial library with a second analyte-analogue and selecting a ligand to which the second analyte-analogue binds, a label including a first component and a second component of a non-radiative fluorescence resonance energy transfer donor-acceptor pair, the analyte-ligand binding pair exhibiting non-radiative fluorescence resonance energy transfer when the first analyte-analogue is bound to the analyte binding ligand, and a change in non-radiative fluorescence resonance energy transfer when the first analyte-analogue is not bound to the analyte binding ligand. In one embodiment, the analyte binding ligand and the analyte analogue are reversibly bound to each other.

In other embodiments, the sensor further includes a matrix surrounding the analyte ligand binding pair. In some embodiments, the sensor further includes a semi-permeable membrane surrounding the analyte ligand binding pair.

In a ninth aspect, the invention features a kit including a sensor described herein.

In a tenth aspect, the invention features a method of making a sensor, the method including selecting an analyte-analogue, attaching a label including a first component of a non-radiative fluorescence resonance energy transfer donor-acceptor pair to an analyte-analogue, selecting an analyte binding ligand from a combinatorial library, the analyte binding ligand being capable of binding with the analyte-analogue, attaching a label including a second component of a non-radiative fluorescence resonance energy transfer donor-acceptor pair to the analyte binding ligand, and encapsulating the labeled analyte binding ligand and the labeled analyte-analogue, the sensor being exhibiting non-radiative fluorescence resonance energy transfer when the analyte-analogue is bound to the analyte binding ligand, and a change in non-radiative fluorescence resonance energy transfer when the analyte-analogue is not bound to the analyte binding ligand.

The invention features a mechanism that decreases the necessity of developing “tailor-made” fluorescent biosensors for analytes. The invention uses combinatorial techniques to identify appropriate ligands for a particular analyte and the sensitivity of FRET techniques to detect analyte-ligand binding. The use of FRET and the ability to select the components of the FRET donor-acceptor label overcomes problems related to wavelength.

The invention also enables the standardization of the selection of the analyte binding ligand and can therefore speed the selection of the analyte binding ligand. The invention also enables the skilled artisan to select analyte binding ligands with affinities for the analyte that fall in a range relevant to the particular sensing application. The invention further enables the analyte binding ligand and the analyte-analogue to be standardized for a given class or classes of analytes, which has the effect of standardizing the labeling method or requirements to achieve an effective FRET signal. These capabilities facilitate and speed up the amount of time required to develop FRET-based sensors for a particular analyte or family of analytes.

The invention also enables the use of a wide choice of fluorescent dyes and a corresponding variety of wavelengths, which increases a user's options with respect to the development and use of FRET-based sensors and allows the user to work in and select from a wider region of potential fluorophores with which to create assays and sensors that employ a wider region of the electromagnetic spectrum. This capability provides the further advantage of enabling measurement at a multitude of wavelengths thereby enabling multiple simultaneous FRET assays of different analytes to be performed without physical separation of the analytes and their analyte binding ligands.

Other features and advantages will be apparent from the following description of the preferred embodiments and from the claims.

### GLOSSARY

In reference to the invention, these terms have the meanings set forth below:

As used herein, “ligand” refers to a molecule that can selectively bind to a receptor molecule or moiety on a receptor molecule. The term “selectively” means that the binding interaction can be detected by a quantifiable assay in the presence of the background signal of non-specific or much weaker interactions. A ligand can be essentially any type of molecule such as a peptide, polypeptide, protein, oligonucleic acid, polynucleic acid, carbohydrate, lipid, or any organic compound. A ligand can also be a combined molecule such as a proteolipid, glyocolipid, glyocopeptide or glycoprotein. Derivatives, analogues and mimetic compounds are intended to be included within the definition of this term, including the addition of metals or other inorganic molecules. A ligand can be multipartite, comprising multiple ligands capable of binding to different sites on one or more receptor molecules. The ligand components of a multi-partite ligand are joined together by an expansion linker. The term ligand therefore refers both to a molecule capable of binding to a receptor molecule and to a portion of such a molecule, if that portion of a molecule is capable of binding to a receptor molecule.

As used herein, “analyte binding ligand” refers to a ligand that binds the analyte of interest.

As used herein, “analogue” refers to a material that has at least some binding properties in common with those of the analyte such that there are ligands that bind to both. The analogue and the analyte do not bind to each other. The analogue may be a derivative of the analyte such as a compound prepared by introducing functional chemical groups onto the analyte that do not affect at least some of the binding properties of the analyte. Another example of a derivative is a lower molecular weight version of the analyte that retains at least some of the binding properties of the analyte.

As used herein, “analyte-analogue,” refers to the analyte, as well as an analogue of the analyte.

As used herein, “analyte-ligand binding pair,” refers to an analyte-analogue and an analyte binding ligand that bind to each other.

As used herein, “reversible binding,” refers to a level of affinity (i.e., the ratio of the forward rate constant to the reverse rate constant) of the analyte-analogue for the analyte binding ligand in a physiological environment or in an environment other than a physiological environment that is sufficient to permit competition between an analyte of interest and the analyte-analogue for the available sites on the analyte binding ligand.

As used herein, “fluorescence” refers to radiation emitted in response to excitation by radiation of a particular set of wavelengths. It includes both short-lived (i.e., in the range of nanoseconds or faster) and longer-lived excited state lifetimes; the latter is sometimes referred to as phosphorescence.

As used herein, “fluorophore” refers to a molecule that accepts radiant energy of one set of wavelengths and emits radiant energy of a second set of wavelengths.

As used herein, “FRET,” refers to non-radiative fluorescence resonance energy transfer.

As used herein, “FRET donor-acceptor pair,” refers to at least two components, e.g., molecules, that exhibit non-radiative fluorescence resonance energy transfer when present in sufficiently close proximity to one another.

As used herein “combinatorial library” refers to a collection of diverse chemical compounds generated by either chemical synthesis or biological synthesis (e.g., in vivo and in vitro biological synthesis) by combining a number of chemical subunits. The subunits may be selected from natural moieties, unnatural moieties and combinations thereof including, e.g., amino acids, nucleotides, sugars, lipids, carbohydrates, synthetic monomer units, synthetic organic monomer units, organic monomer units, and combinations thereof. The compounds of the combinatorial library differ in one or more ways with respect to the number, order, type or types of or modifications made to one or more of the subunits comprising the compounds. A linear combinatorial chemical library such as a polypeptide library is formed by combining a set of chemical building blocks called amino acids in up to every possible way for a given compound length (i.e., the number of amino acids in a polypeptide compound). Millions of chemical compounds can be synthesized through such combinatorial mixing of chemical building blocks. The systematic, combinatorial mixing of 100 interchangeable chemical building blocks results in the theoretical synthesis of 100 million tetrameric compounds or 10 billion pentameric compounds. In general, if there are m possible building blocks forming a linear combinatorial library of length n, then there will be mⁿ potential compounds in the library.

### BRIEF DESCRIPTION OF THE DRAWINGS

FIG. 1A is a graphic representation of absorbance and emission spectra of donor and acceptor molecules.

FIG. 1B is a representation of non-radiative energy transfer.

FIGS. 2a-c illustrate a system that includes components of a FRET-based sensor disposed in a changing environment.

FIGS. 3a-e illustrate an example of a method of screening a combinatorial library.

### DETAILED DESCRIPTION

The invention provides methods of identifying analyte-ligand binding pairs that are capable of exhibiting non-radiative fluorescence resonance energy transfer (i.e., FRET). The invention also provides methods of identifying analyte-ligand binding pairs that are suitable for use in a sensor that operates on the basis of FRET.

I. Principles of FRET

FRET generally involves the non-radiative transfer of energy between two fluorophores, one an energy donor (D) and the other an energy acceptor (A). Any appropriately selected donor-acceptor pair can be used, provided that the emission of the donor overlaps with the excitation spectra of the acceptor and both members can absorb light energy at one wavelength and emit light energy of a different wavelength. Alternatively, both the donor and acceptor can absorb light energy, but only one of the two emits light energy. For example, the donor can be fluorescent and the acceptor can be nonfluorescent, and vice versa. It is also possible to make use of a donor-acceptor pair in which the acceptor is not normally excited at the wavelength used to excite the donor; however, non-radiative FRET causes acceptor excitation.

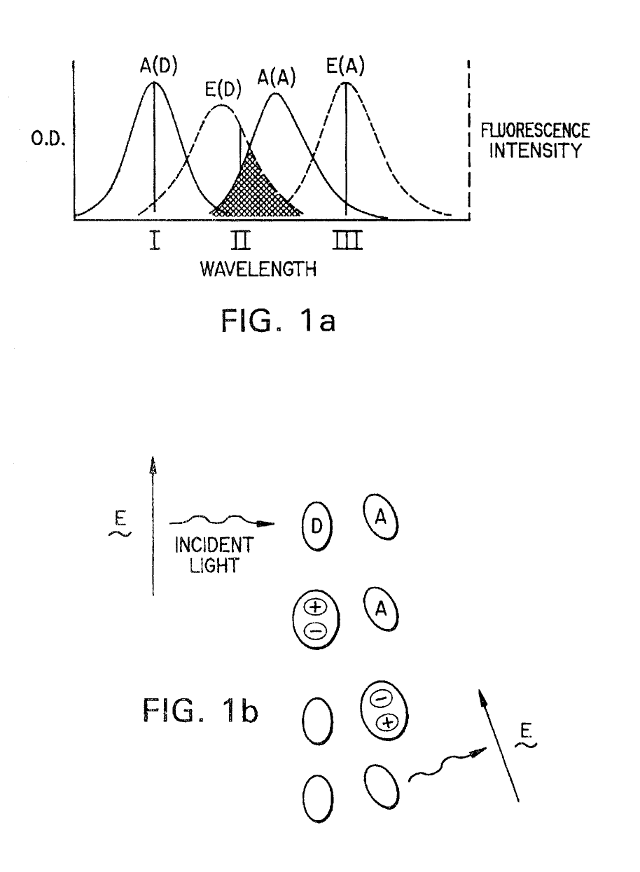

The concept of FRET is represented in FIGS. 1A and 1B. The absorbance and emission of donor, which is designated A(D) and E(D), respectively, and the absorbance and emission of acceptor, which is designated A(A) and E(A), respectively, are represented graphically in FIG. 1A. The area of overlap between the donor emission and the acceptor absorbance spectra (which is the overlap integral) is of importance. If excitation occurs at wavelength I, light will be emitted at wavelength II by the donor, but not at wavelength III by the acceptor because the acceptor does not absorb light at wavelength I.

The non-radiative transfer process that occurs is represented in FIG. 1B. D molecule absorbs the photon whose electric field vector is represented by E. The excited state of D is shown as a dipole with positive charge on one side and negative charge on the other. If an acceptor molecule (A) is sufficiently close to D (e.g., typically less than 100 Angstroms), an oppositely charged dipole is induced on it (it is raised to an excited state). This dipole-induced dipole interaction falls off inversely as the sixth power of donor-acceptor intermolecular distance.

Classically, partial energy transfer can occur. However, this is not what occurs in FRET, which is an all or nothing quantum mechanical event. That is, a donor is not able to give part of its energy to an acceptor. All of the energy must be transferred and energy transfer can occur only if the energy levels (i.e., the spectra) overlap. Energy transfer is an all or nothing probabilistic quantum mechanical event on a molecule by molecule basis. When A leaves its excited state, the emitted light is rotated or depolarized with respect to the incident light. As a result, FRET manifests itself as a decrease in fluorescence intensity (i.e., decrease in donor emission) at II, an appearance of fluorescence intensity at III (i.e., an increase in sensitized emission) and a depolarization of the fluorescence relative to the incident light.

A final manifestation of FRET is in the excited state lifetime. Fluorescence can be seen as an equilibrium process, in which the length of time a molecule remains in its excited state is a result of competition between the rate at which it is being driven into this state by the incident light and the sum of the rates driving it out of this state (fluorescence and non-radiative processes). If a further non-radiative process, FRET, is added (leaving all else unchanged), decay is favored, which means donor lifetime at II is shortened.

When two fluorophores whose excitation and emission spectra overlap are in sufficiently close proximity, the excited state energy of the donor molecule is transferred by a resonance dipole-induced dipole interaction to the neighboring acceptor fluorophore. In FRET, a sample or mixture is illuminated at a wavelength that excites the donor but ideally not the acceptor molecule directly. In practice, a small amount of direct acceptor excitation is acceptable. The sample is then monitored at two wavelengths, i.e., the wavelength of the donor emissions and the wavelength of the acceptor emissions. If donor and acceptor are not in sufficiently close proximity, FRET does not occur and emissions occur only at the donor wavelengths. If donor and acceptor are in sufficiently close proximity, FRET occurs. The results of this interaction are a decrease in donor lifetime, a quenching of donor fluorescence, an enhancement of acceptor fluorescence intensity, and depolarization of fluorescence intensity. The efficiency of energy transfer, Et falls off rapidly as the distance between donor and acceptor molecule, R, increases. For an isolated donor-acceptor pair, the efficiency of energy transfer, assuming a dipole-dipole interaction, is expressed as:

Eₜ=1/[1+(R/Rₒ)⁶] (1)

where R is the separation distance between donor and acceptor and Rₒ is the distance for half transfer. Rₒ is a value that depends upon the overlap integral of the donor emission spectrum and the acceptor excitation spectrum, the index of refraction, the quantum yield of the donor, and the orientation of the donor emission and the acceptor absorbance moments. See, e.g., Forster, T., Z Naturforsch 4A, 321-327 (1949); Forster, T., Disc. Faraday So. 27, 7-17 (1959).

Because of its 1/R⁶ dependence, FRET is extremely dependent on molecular distances and has been dubbed “the spectroscopic ruler”. See, e.g., Stryer, L., and Haugland, R. P., Proc. Natl. Acad. Sci. USA, 98:719 (1967). For example, the technique has been useful in determining the distances between donors and acceptors for both intrinsic and extrinsic fluorophores in a variety of polymers including proteins and nucleic acids. Cardullo et al. demonstrated that the hybridization of two oligodeoxynucleotides could be monitored using FRET. See, e.g., Cardullo, R., et al., Proc. Natl. Acad. Sci., 85:8790-8794 (1988).

The above description of FRET assumes transfer between two singlet states via a dipole-dipole interaction. FRET is not confined to singlet-singlet or dipole-dipole interactions. FRET can occur between singlet- and higher order states such as triplet states, and between higher order states and other higher order states. Similarly FRET can occur via dipole-higher order pole interactions, and via higher pole—higher pole interactions.

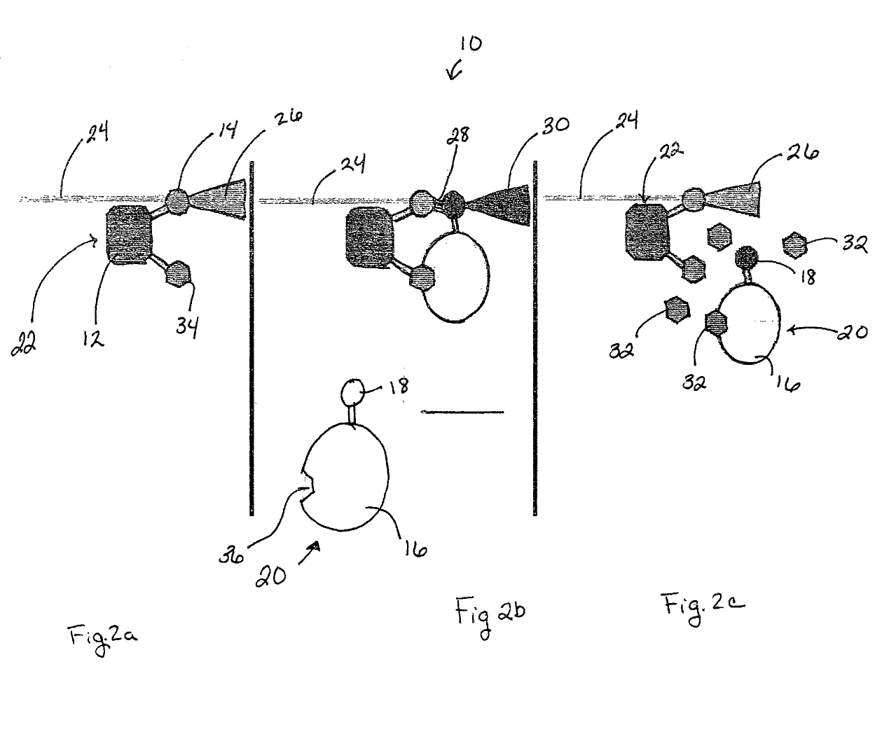

FIGS. 2a-c illustrate a system 10 that includes components of one example of a FRET-based sensor disposed in a changing environment. The FRET-based sensor includes an analyte-analogue 12 that includes a donor fluorophore 14 label and an analyte epitope 34, and an analyte binding ligand 16 that includes an acceptor fluorophore 18 label and an analyte epitope binding site 36. When the fluorophore labeled analyte-analogue (flAA) 22 is not attached to the fluorophore labeled analyte binding ligand (flABL) 20 and is excited by energy of a first wavelength 24, the flAA 22 emits light of a second wavelength 26. When the flAA 22 is bound to the flABL 20 and excitation energy of a first wavelength 24 is transmitted to the pair 22,20, the energy emitted 28 by the flAA 22 is transferred from the donor fluorophore 14 to the acceptor fluorophore 18, whereupon the acceptor fluorophore 18 emits light at a third wavelength 30. As analyte 32 is added to the environment, the flAA 22 flABL 20 complex comes apart, energy transfer decreases, and the donor fluorophore 18 again fluoresces, i.e., emits light, at the second wavelength 26.

Although the donor and the acceptor are referred to herein as a “pair”, the two “members” of the pair can be the same substance. Generally, the two members will be different (e.g., fluorescein and rhodamine). It is possible for one molecule (e.g., fluorescein and rhodamine) to serve as both donor and acceptor; in this case, energy transfer is determined by measuring depolarization of fluorescence. It is also possible for the pair to include more than two members, e.g., two donors and one acceptor.

Examples of useful donor-acceptor pairs include NBD (i.e., N-(7-nitrobenz-2-oxa-1,3-diazol-4-yl)) to rhodamine, NBD to fluorescein to eosin or erythrosine, dansyl to rhodamine, and acrdine orange to rhodamine. Examples of suitable commercially available labels capable of exhibiting FRET include fluorescein to tetramethylrhodamine; 4,4-difluoro-5,7-dimethyl-4-bora-3a,4a-diaza-s-indacene-3-propionic acid, succinimidyl ester, which is commercially available, e.g., under the trade designation BODIPY FL from Molecular Probes (Eugene, Oreg.) to 4,4-difluoro-5-phenyl-4-bora-3a,4a-diaza-sindacene-3-propionic acid, succinimidyl ester, which is commercially available, e.g., under the trade designation BODIPY R6G from Molecular Probes; Cy3.5 monofunctional NHS-ester to Cy5.5 monofunctional NHS-ester, Cy3 monofunctional NHS-ester to Cy5 monfunctional NHS-ester, and Cy5 monofunctional NHS-ester to Cy7 monfunctional NHS-ester, all of which are commercially available from Amersham Biosciences (Buckinghamshire, England); and ALEXA FLUOR 555 carboxylic acid, succinimidyl ester to ALEXA FLUOR 647 carboxylic acid, succinimidyl ester, which are commercially available from Molecular Probes.

Useful protocols for labeling proteins and other biomolecules with FRET donor-acceptor pairs can be found in, e.g., R. Haugland, Handbook of Fluorescent Probes and Research Chemicals (Sixth Ed. 1995) and G. T. Hermanson, Bioconjugate Techniques (1996), and incorporated herein.

II. Method for Determining Analyte-Ligand Binding Pairs Capable of Exhibiting Non-Radiative Fluorescence Resonance Energy Transfer

The method includes obtaining a predetermined analyte binding ligand from a combinatorial library, and labeling at least one of the analyte binding ligand and an analyte-analogue with the components of a FRET donor-acceptor pair such that non-radiative fluorescence resonance energy transfer occurs when the analyte-analogue is bound to the analyte binding ligand, and a change, in non-radiative fluorescence resonance energy transfer occurs when the analyte-analogue is not bound to the analyte binding ligand. The binding pair that forms when the analyte-analogue binds to the analyte binding ligand is hereinafter referred to as the “analyte-ligand binding pair.” The change can be a decrease in, an increase in, or complete loss of, non-radiative fluorescence resonance energy transfer.

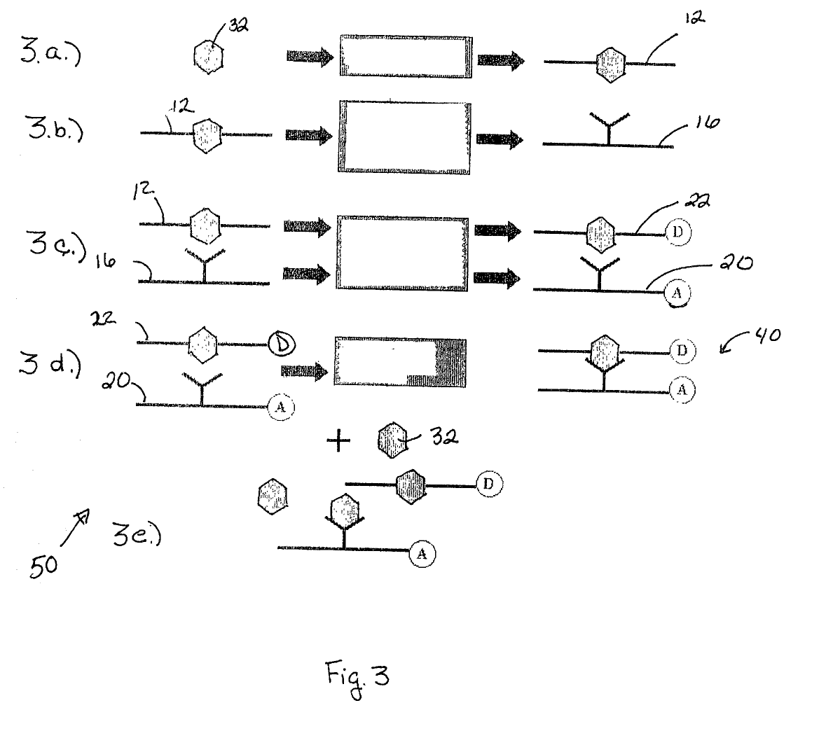

FIGS. 3a-e, illustrate a method of screening a combinatorial library. In FIG. 3a) an analyte 32 is modified to create an analyte-analogue 12. In FIG. 3b) the analyte-analogue is used to screen a combinatorial library for analyte binding ligands 16. In FIG. 3c) the analyte-analogue 12 is labeled with a FRET donor (D) to create a donor labeled analyte-analogue 22, and the analyte binding ligand 16 is labeled with a FRET acceptor (A). In FIG. 3d) donor labeled analogue 22 and acceptor labeled analyte binding ligand 20 are combined and FRET is measured. In FIG. 3e) the addition of analyte 32 results in separation of donor labeled analyte 22 and acceptor labeled analyte binding ligand 20 and reduces the amount of FRET measured as explained in reference to FIG. 2.

A. Identifying the Analyte Binding Ligand

The predetermined analyte binding ligand is identified as being suitable for binding an analyte of interest through the use of a combinatorial library. A combinatorial library of ligands is screened by contacting the library with an analogue to an analyte of interest and identifying at least one ligand that binds the analyte-analogue. The analyte-analogue that is used to screen the combinatorial library and identify an analyte binding ligand may or may not be the same, i.e., have the same chemical structure, as the analyte-analogue used to form the analyte-ligand binding pair.

Preferably at least one ligand of the combinatorial library binds the analyte-analogue. A ligand that binds the analyte or analyte-analogue is referred to herein as the “analyte binding ligand.” If at least one ligand does not bind the analyte-analogue, additional combinatorial libraries are screened until a suitable analyte binding ligand is identified.

The combinatorial library can be selected based upon a variety of factors including, e.g., the nature of the analyte, the level of knowledge about the analyte, known ligands that bind the analyte, and combinations thereof. Useful combinatorial libraries include, e.g., peptide libraries, antibody libraries, apatamer libraries, polynucleic acid libraries including, e.g., deoxyribonucleic acid (DNA) libraries, and ribonucleic acid (RNA) libraries, and synthetic polymer libraries (i.e., libraries of polymers that are derived from more than one type of monomer).

The ligands of a combinatorial library can be constructed to include at least one variable region and at least one constant region. The variable region on the ligands of the combinatorial library represent the site or sites on the ligand that are potentially capable of binding the analyte-analogue. The constant region on the ligands of the combinatorial library preferably includes a region that has been predetermined to be capable of exhibiting a predetermined property, capable of providing a predetermined function, or a combination thereof, including, e.g., being capable of attaching, preferably covalently, at least one component of a FRET donor-acceptor pair. The constant region can be referred to as the FRET binding site. Suitable FRET binding sites include those regions positioned on the molecule such that when a FRET label is attached thereto FRET occurs. Techniques for determining the suitable placement of the components of the FRET donor acceptor pairs on a molecule are described in various literature sources including, e.g., Cardullo, R., et al., Proc. Natl. Acad. Sci., 85:8790-8794 (1988), and Richard Haugland “Handbook of Fluorescent Probes and Research Products Ninth Edition Molecular Probes, Eugene, Oreg. 2002), and incorporated herein.

Alternatively or in addition, the constant region of the ligand can include a component of the FRET donor-acceptor pair. Combinatorial libraries constructed to include such a constant region include, e.g., peptide libraries constructed from a random peptide sequence that is preceded or followed by a constant region that includes nucleic acid or lysine labeled with a fluorophore, peptide libraries synthesized to include amino acids or amino acid analogues labeled with fluorophores including, e.g., γ-EDANS-α-9-fluorenylmethoxy-carbonyl, L-glutamic acid (commercially available from Molecular Probes), Nα-9-fluorenylmethoxy-carbonyl, Nα-7-nitrobenz-2-oxa-1,2,-diazol-4-yl,L-diaminopropionic acid (as described in, e.g., Dufau, I., and Mazarguil, H. (2000) “Design of a fluorescent amino acid derivative useful in peptide synthesis,” Tetrahedron. Lett., 41, 6063-6066), nucleic acid libraries can be constructed to include a constant region that includes a fluorescent moiety by, e.g., incorporating a fluorescent moiety into the nucleic acid sequence, labeling the nucleic acid with a fluorophore, or incorporating a green fluorescent protein in the structure of the nucleic acid ligand.

Methods for incorporating a constant region into a ligand of a combinatorial library are described in various literature sources including, e.g., Vaughn et al., Nature Biotechnology, 14(3):309-314 (1996)), PCT Patent Application No. US96/10287, M. Famulok, E. L. Winnacker, and C. H. Wong eds., Current Topics in Microbiology and Immunology Springer, Verlag, Bonn, Germany, 243: 87-105 (1999), and Shmuel Cabilly, “The Basic Structure of Filamentous Phage and its Use in the Display of Combinatorial Peptide Libraries,” Methods in Molecular Biology, vol. 87: Combinatorial Peptide Library Protocols (S. Cabilly Humana Press Inc., Totwa, N.J.) (pages 129-136) (1998), and incorporated herein.

Other useful combinatorial libraries include ligands that are labeled with at least one component of a FRET donor-acceptor pair.

A combinatorial library that includes at least one component of a FRET donor-acceptor pair, whether through labeling of the ligand of the library with FRET donor-acceptor pair or through incorporation of the component of the a FRET donor-acceptor pair into the structure of the ligand, enables a simultaneous determination of both the presence of an analyte binding ligand and FRET, if desired. The simultaneous determination of the presence of an analyte binding ligand and FRET can be achieved, for example, by labeling the analyte-analogue with a second component of a FRET donor-acceptor pair. When the FRET-labeled analyte-analogue is brought into contact with the FRET-labeled combinatorial library, the presence of FRET indicates that the analyte-analogue is bound to a ligand and that the binding pair is capable of producing FRET. Alternatively two components of the FRET donor-acceptor pair can be attached to or incorporated in the ligands of the library.

Various methods of preparing combinatorial libraries and screening combinatorial libraries to identify binding pairs are available. These methods are well known to those skilled in the art and include, e.g., solid phase synthesis (e.g., bead method), phage display, and phage expression. Methods of making combinatorial libraries are described in various patent and literature sources including, e.g., Advanced ChemTech Handbook of Combinatorial & Solid Phase Organic Chemistry, (pages 7-34) (1998); K. Johnsson and L. Ge, “Phage Display of Combinatorial Peptide and Protein libraries and their Applications in Biology and Chemistry,” Combinatorial Chemistry in Biology, M. Famulok, E. L. Winnacker, and C. H. Wong eds., Current Topics in Microbiology and Immunology Springer, Verlag, Bonn, Germany, 243: 87-105 (1999); Kit S. Lam, Michal Lebl, “Synthesis of One-Bead one-Compound Combinatorial Peptide Library,” Methods in Molecular Biology, vol. 87: Combinatorial Peptide Library Protocols (S. Cabilly Humana Press Inc., Totwa, N.J. (pages 1-6) (1998); Shmuel Cabilly, “The Basic Structure of Filamentous Phage and Its Use in the Display of Combinatorial Peptide Libraries,” Methods in Molecular Biology, vol. 87: Combinatorial Peptide Library Protocols (S. Cabilly Humana Press Inc., Totwa, N.J. (pages 129-136) (1998); M. Famulok, E. L. Winnacker, and C. H. Wong, Combinatorial Chemistry in Biology, M. Famulok and G. Mayer, “Aptamers as Tools in Molecular Biology and Immunology,” (pages 123-136 (1999)), and incorporated herein.

Useful screening techniques include the techniques described in sources including, e.g., Shmuel Cabilly, Judith Heldman, and Ephraim Katchalski-Katzir, “Screening Phage Display Peptide Libraries on Nitrocellulose Membranes,” Methods in Molecular Biology, vol. 87: Combinatorial Peptide Library Protocols, Chapter 20, (S. Cabilly Humana Press Inc., Totwa, N.J. (pages 185-194) (1998); M. Famulok, E. L. Winnacker, and C. H. Wong, Combinatorial Chemistry in Biology, J. Hanes and A. Plückthun, “In Vitro Selection Methods for Screening of Peptide and Protein Libraries,” (pages 107-122) (1999).

Useful combinatorial chemical libraries include, e.g., peptide libraries as described in, e.g., U.S. Pat. No. 5,010,175, Furka, Int. J. Pept. Prot. Res., 37: 487-493 (1991), and Houghton et al. Nature, 354: 84-88 (1991)); peptoids as described in, e.g., PCT Publication No. WO 91/19735, Dec. 26, 1991; encoded peptides as described in, e.g., PCT Publication No. WO 93/20242, Oct. 14, 1993; random bio-oligomers as described in, e.g., PCT Publication No. WO 92/00091, Jan. 9, 1992; benzodiazepines as described in, e.g., U.S. Pat. No. 5,288,514; diversomers including, e.g., hydantoins, benzodiazepines and dipeptides as described in, e.g., Hobbs et al., Proc. Nat. Acad. Sci. USA 90: 6909-6913 (1993)); vinylogous polypeptides as described in, e.g., Hagihara et al., J. Amer. Chem. Soc. 114: 6568 (1992); nonpeptidal peptidomimetics with a Beta-D-Glucose scaffolding as described in, e.g., Hirschmann et al., J. Amer. Chem. Soc. 114: 9217-9218 (1992); organic synthesis of small compound libraries are described in, e.g., Chen et al., J. Amer. Chem. Soc. 116:2661(1994)); oligocarbamates described in, e.g., Cho, et al., Science 261:1303 (1993)); peptidyl phosphonates (Campbell et al., J. Org. Chem. 59:658 (1994) and Gordon et al., J. Med. Chem. 37:1385 (1994), nucleic acid libraries, which are commercially available, e.g., from Strategene, Corp., peptide nucleic acid libraries as described in, e.g., U.S. Pat. No. 5,539,083, antibody libraries as described in, e.g., in Vaughn et al., Nature Biotechnology, 14(3):309-314 (1996) and PCT Application No. US96/10287, carbohydrate libraries as described in, e.g., Liang et al., Science, 274:1520-1522 (1996), and U.S. Pat. No. 5,593,853, and small organic molecule libraries including, e.g., benzodiazepines as described in, e.g., Baum, C&EN, January 18, page 33 (1993), isoprenoids as described in, e.g., U.S. Pat. No. 5,569,588, thiazolidinones and metathiazanones as described in, e.g., U.S. Pat. No. 5,549,974, pyrrolidines as described in, e.g., U.S. Pat. Nos. 5,525,735 and 5,519,134, morpholino compounds as described in, e.g., U.S. Pat. No. 5,506,337, and benzodiazepines as described in, e.g., U.S. Pat. No. 5,288,514), and incorporated herein.

Devices for preparing combinatorial libraries are commercially available and include, e.g., 357 MPS, 390 MPS, Advanced Chem Tech (Louisville Ky.), Symphony, Rainin (Woburn, Mass.), 433A Applied Biosystems (Foster City, Calif.), and 9050 Plus, Millipore (Bedford, Mass.).

A number of robotic systems have also been developed for solution phase chemistries. These systems include automated workstations including, e.g., the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.).

Suitable commercially available combinatorial libraries include, e.g., combinatorial libraries commercially available from Advanced ChemTech (Louisville, Ky.), ComGenex, (Princeton, N.J.), Asinex (Moscow, Russia), Tripos, Inc. (St. Louis, Mo.), ChemStar, Ltd, (Moscow, Russia), 3D Pharmaceuticals (Exton, Pa.), Phylos (Lexington, Mass.), Cambridge Antibody Technology (Cambridge, United Kingdom), MorphSys (Munich, Germany), and Martek Biosciences (Columbia, Md.).

B. Determining Affinity

After an analyte binding ligand is identified, the analyte-ligand binding pair is preferably screened to determine the level of affinity the analyte-analogue has for the analyte binding ligand. The preferred level of affinity is usually dependent on the application in which the analyte-ligand binding pair is to be used. In the case in which the analyte-ligand binding pair is to be used in a competitive assay, it is preferable that a suitable level of competition exists between the analyte-analogue and the analyte for the analyte binding site(s) on the analyte binding ligand such that the level of analyte present in the environment surrounding the analyte-ligand binding pair can be determined based upon the displacement of the analyte-analogue from the analyte binding ligand.

Competitive assays generally involve the competition between the analyte present in a sample and an analyte-analogue for a limited number of binding sites on the analyte binding ligand(s). Useful competitive assays include homogeneous and heterogeneous competitive assays. In homogeneous assays, all of the reactants participating in the competition are mixed together and the quantity of analyte is determined by its effect on the extent of binding between analyte binding ligand and the analyte-analogue without separating bound and unbound analyte analogue. In heterogeneous assays, the amount of analyte-analogue bound to analyte binding ligand is determined after separation of bound analyte anlogue from free analyte analogue.

Various methods are available for studying the affinity of an analyte-analogue for an analyte binding ligand. Assays for determining affinity can be carried out in solution using direct binding techniques or competition binding techniques and detection tracers such as fluorescence or radioactivity. Direct binding assays measure the specific fraction of labeled bound analyte binding ligand to analyte-analogue. Competition binding assays infer the fraction of bound analyte binding ligand to analyte-analogue by measuring the displacement of a labeled analyte binding ligand from the analyte-analogue by an inhibitor, e.g., analyte. In each case, bound analyte binding ligand is separated from unbound analyte binding ligand using methods such as equilibrium dialysis, filtration, size-exclusion column chromatography, centrifugation and combinations thereof as described, e.g., in L. E. Limbird, Cell Surface Receptors: A short course on theory and methods (1986). Upon analysis of the results of a direct binding assay, the total number of analyte-analogue binding sites and the equilibrium binding affinity (K_{D}) of the analyte binding ligand to the analyte-analogue are determined. The analysis of a competition binding assay also identifies the concentration at which 50% of the available sites on the analyte binding ligand are occupied by the analyte-analogue or inhibitor, e.g., analyte. This concentration is referred as the inhibitor concentration (IC) that causes a 50% maximal effect, i.e., IC₅₀ The IC₅₀ can be converted to an equilibrium binding affinity value (K₁) using the Cheng-Prusoff relationship as described, e.g., in Cheng Y., and Prusoff, W. H., “Relationship Between the Inhibition Constant (K₁) and the Concentration of an Inhibitor that Causes a 50% Inhibition (IC₅₀) of an Enzymatic Reaction,” Biochem. Pharacol. 22:3099 (1973).

Homogenous assay methods can be used to determine the binding affinity an analyte-analogue has to an analyte binding ligand. Homogenous assays do not require separation of bound from unbound analyte binding ligand. These methods are limited to fluorescent detection tracers and can be measured in both direct binding and competition binding assays using monitoring techniques such as FRET, Fluorescence Polarization and Fluorescence Correlation Spectroscopy.

C. Analyte-Analogue

The analogue of the analyte (i.e., the analyte-analogue) can be a modified analyte, as well as a fragmented or synthetic portion of the analyte molecule, provided the analyte-analogue has at least one epitopic site in common with the analyte of interest. Any possible analyte-analogue may be suitable. Where the analyte is a protein or a peptide, an example of a suitable analyte-analogue is a synthetic peptide sequence that duplicates at least one epitope of the whole-molecule analyte so that the analyte-analogue can bind to an analyte-specific binding member. Where the analyte is an organic molecule, an example of a suitable analyte-analogue is a protein or peptide to which the analyte is covalently attached. Where glucose is the analyte, suitable analyte-analogues include, e.g., glycosylated human serum albumin, and glycosylated albumin as described, e.g., in U.S. Pat. No. 6,040,194.

Other suitable analyte-analogues include those analyte-analogues engineered to contain a second epitope that contains a tag binding site. For example, peptides, proteins, and oligonucleotides can be synthesized with a biotin epitope to form a biotinylated analyte-analogue. These biotin-modified analyte analogues will recognize and bind to streptavidin, which may be labeled with a component of a FRET donor-acceptor pair. Another suitable analyte-analogue include peptide and protein analyte analogues in which a hapten, e.g., dinitrophenyl or nitrotyrosine, has been incorporated. Specific fluorescent anti-hapten antibodies will then bind to the haptenated analyte analogue.

Other useful analogues to analytes such as polynucleotides include, e.g., fluorescently labeled oligonucleic acids, which are described in, e.g., Cardullo, R., et al., Proc. Natl. Acad. Sci., 85:8790-8794 (1988), and Richard Haugland “Handbook of Fluorescent Probes and Research Products Ninth Edition, Molecular Probes, Eugene, Oreg. 2002, and incorporated herein, and oligonucleic acids attached to peptides and proteins.

Alternatively or in addition, the analogue to the analyte can include one or more components of the FRET label. In the case where the analogue is an analyte labeled with a component of the FRET donor-acceptor pair, it is often attached to the analyte through a linking moiety that, in some cases, includes a spacer. Spacers can provide a number of functions including, e.g., providing physical space or clearance between the FRET label and the analyte epitope such that the component of the FRET label does not interfere with the interaction of the analyte binding ligand with the analyte epitope, providing sufficient segmental flexibility so as to result in efficient FRET, and combinations thereof. Useful reactive fluorophores that create linking moieties include, e.g., 6-carboxyfluorescein, succinimidyl ester, which is commercially available, e.g., under the trade designation C6164 from Molecular Probes, and 6-(fluorescein-5-carboxamido) hexanoic acid, succinimidyl ester, which is commercially available, e.g., under the trade designation F-6106 from Molecular Probes. Useful spacers include, e.g., methylene and peptide chains.

The linking moiety can be attached to a component of the FRET label, the analyte-analogue or the analyte binding ligand. Attaching the linking moiety can occur in any desired sequence including, e.g., first attaching the linking moiety to the analyte binding ligand or analyte-analogue and then attaching a component of the FRET label to the linking moiety, attaching the linking moiety to a component of the FRET label and then attaching the linking moiety to the analyte binding ligand or analyte-analogue, and combinations thereof.

The analyte-analogue preferably includes a region that has been predetermined to be suitable for binding at least one component of a FRET donor-acceptor pair. Once a region for binding at least one component of a FRET donor-acceptor pair is determined, the skilled artisan can create analogues to other analytes with knowledge that if the analogue includes the predetermined region (i.e., the FRET-label binding site) it will likely be capable of binding the same component of the FRET donor-acceptor pair, and upon interaction with analyte binding ligands selected from a particular class of combinatorial assay, the spatial relationship between donor and acceptor elements of the FRET pair will be such as to promote FRET. In other words, by determining the binding site or region on the analogue that is capable of binding a component of a FRET donor-acceptor pair, the analogue can be standardized for use with other analytes.

The analyte-analogue optionally can be labeled with at least one component of a FRET donor-acceptor pair prior to contact of the analyte-analogue with the combinatorial library. As indicated above, if the analyte-analogue and the ligands of the combinatorial library both include a component of the FRET donor-acceptor pair, successful binding of analyte-analogue to analyte binding ligand can be determined by the presence of FRET.

D. Labeling Moieties with a FRET Donor-Acceptor Pair

The component(s) of the FRET donor-acceptor pair (i.e., the FRET-label) can be attached to the analyte-analogue, the analyte binding ligand or a combination thereof. Preferably the analyte binding ligand is labeled with a first component of the FRET donor-acceptor pair and the analyte-analogue is labeled with a second component of the FRET donor-acceptor pair. Alternatively, there can be two analyte-analogues capable of attachment to a single analyte binding ligand. The two analyte-analogues can each be labeled with a component of the FRET donor-acceptor pair such that when the two components are in sufficiently close relation to each other, e.g., when bound to sites on the analtye binding ligand, FRET occurs.

The FRET labels are attached to the components of the analyte-ligand binding pair in such a way that when the analyte-analogue is bound to the analyte binding ligand, non-radiative fluorescence resonance energy transfer occurs, and when the analyte-analogue is not bound to the analyte binding ligand, fluorescence energy transfer decreases and preferably dissipates entirely.

In an embodiment in which two components of the FRET label are attached to a single component of the analyte-ligand binding pair, i.e., either the analyte binding ligand or the analyte-analogue, the two components of the FRET donor-acceptor pair are positioned on the component of the analyte-ligand binding pair such that when the analyte-analogue is bound to the analyte binding ligand, the labeled component of the analyte-ligand binding pair assumes an orientation that permits FRET to occur and when the analyte-analogue is not bound to the analyte binding ligand, the labeled component of the analyte-ligand binding pair assumes an orientation such that FRET does not occur.

The analyte binding ligand and the analyte-analogue can be labeled using any suitable method of labeling ligands and analytes with FRET donor-acceptor pairs. A variety of useful FRET labeling methods are known in the art and include, e.g., the labeling of ε amino groups of lysine moieties with either isothiocyanates or succinimidyl esters, labeling the thiol groups on cysteines with maleimides, and those methods disclosed in various literature sources including, e.g., Richard Haugland “Handbook of Fluorescent Probes and Research Products Ninth Edition Molecular Probes, Eugene, Oreg. 2002, and Anthony K. Tong and Jingyue Ju, “Single Nucleiotide Polymorphism Detection by Combinatorial Fluorescence Energy Transfer Tags and Biotinylated Dideoxynucleotides,” Nucleic Acids Research, Vol. 30, No. 5 (2002), and G. T. Hermanson, Bioconjugate Techniques (1996).

The analyte binding ligand, the analyte-analogue and combinations thereof can be labeled with the FRET donor-acceptor pair at any point during the method including, e.g., prior to contact between the combinatorial library and the analyte-analogue, after contact between the combinatorial library and the analyte-analogue, after the analyte binding ligand has been determined but prior to determining the level of affinity between the analyte-analogue and the analyte binding ligand, after an analyte binding ligand has been identified and after determining the level of affinity, and combinations thereof.

In other embodiments, the FRET label is applied to or incorporated in the components of the combinatorial library as the combinatorial library is synthesized. Such techniques include, e.g., synthesizing peptide combinatorial libraries such that they include at least one subunit that is fluorescent, generating antibody combinatorial libraries using cDNA that codes for a naturally fluorescent protein, e.g., from the family of green fluorescent proteins, such that the cDNA sequence for the naturally fluorescent protein is inserted into the cDNA sequence of the constant region of the combinatorial library, and inserting fluoronucleic acids in peptides. Useful methods of applying a FRET label to or incorporating a FRET label in the ligands of a combinatorial library as the combinatorial library is synthesized are described in, e.g., U.S. Pat. Nos. 6,040,194 and 5,491,084, Chalfie and Prasher “Uses of Green-Fluorescent Protein,” Dufau, I., and Mazarguil, H. (2000) “Design of a fluorescent amino acid derivative useful in peptide synthesis,” Tetrahedron. Lett., 41, 6063-6066, and Richard Haugland, “Handbook of Fluorescent Probes and Research Products Ninth Edition Molecular Probes, Eugene, Oreg. 2002, and incorporated herein.

Once the FRET-label binding site has been determined, an analogue containing the same FRET-label binding site can be formed for other analytes including, e.g., analytes of the same class as the first analyte.

III. Method of Using FRET for Analyte Detection

In general, FRET is used for analyte detection in one of two ways. The first is a competitive assay in which the analyte-analogue and the analyte binding ligand are labeled, one with a donor fluorophore and the other with an acceptor fluorophore. The analyte-analogue may be labeled with donor and the analyte binding ligand may be labeled with acceptor. Alternately, the analyte-analogue may be labeled with acceptor and the analyte binding ligand may be labeled with the donor. When the labeled analyte binding ligand and analyte-analogue contact analyte, analyte displaces the analyte-analogue that is bound to the analyte binding ligand. Because the analyte binding ligand and the analyte-analogue are no longer close enough to each other for FRET to occur, the fluorescence signal due to FRET decreases; the decrease correlates with the concentration of analyte (the correlation of the FRET signal and concentration can be established in a prior calibration step).

For applications in which it is desirable to reuse the fluorescence reagents, i.e., the fluorescent labeled analyte binding ligand and analyte-analogue, the binding between analyte and analyte binding ligand preferably is reversible. Similarly, the equilibrium binding constants associated with analyte-ligand binding and analogue-ligand binding preferably is such that analyte can displace analogue. In other words, analogue-ligand binding preferably is not so strong that analyte cannot displace the analyte-analogue.

Preferably the analyte-ligand binding pair exhibits a suitable degree of reversible binding in environments including, e.g., physiological environments, and liquid environments both in vitro and in vivo.

IV. FRET-Based Sensors

The analyte-ligand binding pairs identified in accordance with the methods described herein and FRET donor-acceptor pair labeled derivatives thereof are useful in a variety of sensors capable of sensing the presence of analyte in an environment including. The sensor can be constructed to detect the presence, concentration, or a combination thereof, of analyte in various in vitro and in vivo environments including, e.g., physiological environments including, e.g., body fluids (e.g., blood, urine, saliva, extracellular fluid, peritoneal fluids, and pericardial fluid), and nonphysiological environments including, e.g., liquid, solid, and gaseous samples. The sensor can be constructed to remain active for extended periods of time (e.g., one month or more) before having to be replaced.

The sensors can be in a variety of forms including, e.g., microcapsules, kits, and probes, and is preferably constructed to include a material capable of retaining the FRET-labeled analyte-ligand binding pair at the desired location in the environment in which it is to function, so as to allow contact or communication with the analyte. Suitable sensor constructions include, e.g. the FRET-labeled analyte-ligand binding pair surrounded by a semipermeable membrane, the FRET-labeled analyte-ligand binding pair disposed (e.g., encapsulated) in a matrix (e.g., a spherical matrix), the FRET-labeled analyte-ligand binding pair disposed in a vessel (e.g., a microdialysis vessel), and combinations thereof. Alternatively, the sensor can be constructed such that the FRET-labeled analyte-ligand binding pair is dispersed in an oil, e.g., silicone oil, fluorocarbon oil and combinations thereof. The sensor preferably is constructed to be suitable for implanting anywhere in the body.

Suitable semipermeable membranes allow the passage of substances up to a predetermined size and provide an effective barrier to the passage of substances larger than the predetermined size. The semipermeable membrane preferably has a molecular weight cut off, i.e., the highest molecular weight that is allowed to pass through the membrane, sufficient to maintain the chemistry of the FRET pair in the sensor, allow analyte to move in and out of the sensor, and, optionally, to inhibit and preferably prevent the sensor from eliciting an immune response from a host in which the sensor is implanted. The molecular weight cutoff range can also be selected based on the type and extent of immunological response anticipated for the sensor after the sensor is implanted. The molecular weight cut off range can be a function of the pore size of the semipermeable membrane.

Useful semipermeable membrane materials include polyamino acids including, e.g., polylysine, polyornithine, polyalanine, polyarginine and polyhistidine, chitosan, polyacrylonitrile/polyvinylchloride, polyethylene oxide, polyvinyl acetate, polyacrylonitrile, polymethylmethacrylate, polyvinyldifluoride, polyethylene oxide, polyolefins (e.g., polyisobutylene and polypropylene), polysulfones, cellulose derivatives (e.g., cellulose acetate and cellulose butyrate), and combinations thereof. Suitable semipermeable membranes are described, e.g., in U.S. Pat. Nos. 6,126,936, and 6,368,612, and also include nucleopore membrane technologies available from Whatman (Newton, Mass.).

Suitable semipermeable membranes also result from modifying a portion of the structure of an encapsulation matrix. One method of modifying the structure of the matrix includes crosslinking the matrix using metal ions including, e.g., calcium ions, barium ions, iron ions, chemical crosslinking agents (e.g., gluteraldehyde), and combinations thereof. The degree of crosslinking affects the porosity of the resulting membrane.

Examples of suitable encapsulation matrices include biocompatible gels, e.g., hydrogels, i.e., a three-dimensional network of cross-linked hydrophilic polymers. Suitable hydrogels include, e.g., gels that carry a net negative charge (e.g., alginate), gels that carry a net positive charge including, e.g., extracellular matrix components such as collagen and laminin, gels that include a net neutral charge including, e.g., crosslinked polyethylene oxide and polyvinyl alcohol, and agarose. Suitable extracellular matrix components are commercially available under the trade designation MATRIGEL from Collaborative Biomedical (Bedford, Mass.), and VITROGEN from Cohesion Technologies (Palo Alto, Calif.).

The sensor can be utilized in a variety of techniques including, e.g., placing the FRET-labeled analyte binding ligand pair in, on, or under the skin, in an organ, in a vessel (e.g., a vein or artery), and combinations thereof such that the FRET-labeled analyte binding ligand pair is in communication with (e.g., contacting) the analyte.

In the embodiment in which the FRET-labeled analyte binding ligand pair is positioned in, on or under the skin, the analyte can be detected by illuminating the skin at the donor excitation wavelength and monitoring fluorescence emission at wavelengths characteristic of the donor and acceptor. For example, if the fluorescent materials are fluorescein and rhodamine, fluorescence intensities are monitored at 520 nM and 596 nM (i.e., the respective emission maximum wavelengths). The measure of energy transfer, as detected by a fluorimeter, is then either the ratio of fluorescence intensities at the two emission wavelengths (e.g., 520 nm and 596 nm) or other measure of the relative amounts of donor and acceptor fluorescence (e.g., donor fluorescence liftetime) or the quenching of the donor (e.g., fluorescein) fluorescence at its emission maximum as a function of analyte concentration.

The FRET-labeled analyte-ligand binding pair may also be tattooed onto the skin or contained in a transcutaneous patch. Alternatively, the FRET-labeled analyte-ligand binding pair may be modified in such a way that when injected subcutaneously, it becomes bound to cell structure and remains fixed in situ under the skin.

Alternatively, the FRET-labeled analyte-ligand binding pair can be placed in communication with a sample of body fluid that contains the analyte of interest and that has been removed from the body. For example, the sensor containing the FRET-labeled analyte-ligand binding pair can be used to detect and quantify the analyte of interest by placing the sensor containing the FRET-labeled analyte-ligand binding pair in communication with analyte-containing bodily fluid in a fluorimeter.

Alternatively, the FRET-labeled analyte-ligand binding pair may be adhered to a solid substrate (e.g., a stick) or may be contained in a chamber (e.g., a microdialysis vessel). The FRET-labeled analyte-ligand binding pair may also be contained in a pen cartridge that dispenses an appropriate volume of the FRET-labeled analyte-ligand binding pair into a sample, e.g., blood or other bodily fluid, containing analyte.

Other embodiments are within the scope of the claims. Although the FRET has been described herein with reference to the presence of FRET occurring when the analyte-analogue is bound to the analyte binding ligand, in an alternate embodiment, the absence of FRET can be indicative of the analyte-analogue being bound to the analyte binding ligand.

What is claimed is:

1. A method of identifying an analyte-ligand binding pair that exhibits non-radiative fluorescence resonance energy transfer, said method comprising:
a) obtaining a predetermined analyte binding ligand from a combinatorial library comprising ligands, said analyte binding ligand having been predetermined by contacting the combinatorial library with a first analyte-analogue and selecting a ligand to which the first analyte-analogue binds; and
b) attaching a label to at least one of said analyte binding ligand and a second analyte-analogue, said label comprising at least one of a first component and a second component of a non-radiative fluorescence resonance energy transfer donor-acceptor pair such that
non-radiative fluorescence resonance energy transfer occurs when said second analyte-analogue is bound to said analyte binding ligand, and
a change in non-radiative fluorescence resonance energy transfer occurs when said second analyte-analogue is not bound to said analyte binding ligand.

2. The method of claim 1, wherein, prior to obtaining said predetermined analyte binding ligand, said predetermined analyte binding ligand comprises a label comprising said first component of said non-radiative fluorescence resonance energy transfer donor acceptor pair.

3. The method of claim 2, comprising attaching said second component of said non-radiative fluorescence resonance energy transfer donor-acceptor pair to said second analyte-analogue.

4. The method of claim 1, comprising attaching said first component of said non-radiative fluorescence resonance energy transfer donor-acceptor pair to said analyte binding ligand and attaching said second component of said non-radiative fluorescence resonance energy transfer donor-acceptor pair to said second analyte-analogue.

5. The method of claim 1, wherein said label further comprises a linking moiety attached to said analyte binding ligand and at least one of said first component and said second component of said non-radiative fluorescence resonance energy transfer donor-acceptor pair, said moiety being capable of being bound to said analyte binding ligand and at least one of said first component and said second component of said non-radiative fluorescence resonance energy transfer donor-acceptor pair

6. The method of claim 1, further comprising attaching a linking moiety to at least one of said analyte binding ligand and at least one of said first component and said second component of said non-radiative fluorescence resonance energy transfer donor-acceptor pair, said moiety being capable of being bound to said analyte binding ligand and at least one of said first component and said second component of said non-radiative fluorescence resonance energy transfer donor-acceptor pair.

7. The method of claim 1, comprising attaching said first component and said second component of said non-radiative fluorescence resonance energy transfer donor-acceptor pair to said analyte binding ligand.

8. The method of claim 1, comprising attaching said first component and said second component of said non-radiative fluorescence resonance energy transfer donor-acceptor pair to said second analyte-analogue.

9. The method of claim 1, wherein the combinatorial library comprises a library selected from the group consisting of peptide library, antibody library, antibody fragment library, nucleic acid library, apatamer library, polymer library, and combinations thereof.

10. The method of claim 1, wherein said ligands are selected from the group consisting of polymers, antibodies, antibody fragments, nucleotides, peptides, apatamers, and combinations thereof.

11. The method of claim 1, wherein the second analyte-analogue has the same chemical structure as the first analyte-analogue.

12. The method of claim 1, wherein the second analyte-analogue has a different chemical structure from the first analyte-analogue.

13. A method of identifying an analyte-ligand binding pair that exhibits non-radiative fluorescence resonance energy transfer, said method comprising:
a) contacting a combinatorial library with an analyte-analogue, said combinatorial library comprising ligands;
b) identifying at least one ligand to which said analyte-analogue binds, said ligand being the analyte binding ligand; and
c) attaching a label to at least one of said analyte binding ligand and said analyte-analogue, said label comprising at least one of a first component and a second component of a non-radiative fluorescence resonance energy transfer donor-acceptor pair such that
non-radiative fluorescence resonance energy transfer occurs when said analyte-analogue is bound to said analyte binding ligand, and
a change in non-radiative fluorescence resonance energy transfer occurs when said analyte-analogue is not bound to said analyte binding ligand.

14. The method of claim 13, comprising attaching a first component of a non-radiative fluorescence resonance energy transfer donor-acceptor pair to said analyte binding ligand.

15. The method of claim 13, comprising attaching a first component of a non-radiative fluorescence resonance energy transfer donor-acceptor pair to said ligands of said combinatorial library prior to contacting said combinatorial library with said analyte-analogue.

16. The method of claim 13, comprising attaching a first component of a non-radiative fluorescence resonance energy transfer donor-acceptor pair to said analyte-analogue prior to contacting said combinatorial library with said analyte-analogue.

17. The method of claim 13, comprising attaching said first component and said second component of said non-radiative fluorescence resonance energy transfer donor-acceptor pair to said analyte binding ligand.

18. The method of claim 13, comprising attaching said first component and said second component of said non-radiative fluorescence resonance energy transfer donor-acceptor pair to said ligands of said combinatorial library prior to contacting said combinatorial library with said analyte-analogue.

19. The method of claim 13, comprising attaching said first component and said second component of said non-radiative fluorescence resonance energy transfer donor-acceptor pair to said analyte-analogue.

20. The method of claim 13, comprising attaching said first component and said second component of said non-radiative fluorescence resonance energy transfer donor-acceptor pair to said analyte-analogue prior to contacting said combinatorial library with said analyte-analogue.

21. The method of claim 13, further comprising selecting an analyte binding ligand to which said analyte-analogue exhibits reversible binding.

22. The method of claim 13, wherein the analyte comprises glucose.

23. The method of claim 13, wherein the combinatorial library comprises a library selected from the group consisting of peptide library, antibody library, antibody fragment library, nucleic acid library, apatamer library, polymer library, and combinations thereof.

24. The method of claim 13, wherein said ligands are selected from the group consisting of polymers, antibodies, antibody fragments, nucleotides, peptides, apatamers, and combinations thereof.

25. A method of identifying an analyte-ligand binding pair that exhibits non-radiative fluorescence resonance energy transfer, said method comprising:
a) contacting a combinatorial library comprising a plurality of ligands with an analyte-analogue such that said analyte-analogue binds to at least one of said ligands to form an analyte-ligand binding pair, said ligands comprising a first label comprising a first component of a non-radiative fluorescence resonance energy transfer donor-acceptor pair, at least one of said analyte-analogue and said ligands comprising a second label comprising a second component of a non-radiative fluorescence resonance energy transfer donor-acceptor pair; and
b) detecting an analyte-ligand binding pair that exhibits non-radiative fluorescence resonance energy transfer.

26. The method of claim 25 further comprising identifying said analyte-ligand binding pair.

27. The method of claim 25, wherein said identifying and said detecting occur simultaneously or substantially simultaneously.

28. The method of claim 25, further comprising identifying an analyte-analogue-ligand binding pair that exhibits a change in non-radiative fluorescence resonance energy transfer in the presence of analyte.

29. The method of claim 25, wherein at least one of the first and second components of the non-radiative fluorescence resonance energy transfer donor acceptor pair is selected from the family of green fluorescent proteins.

30. The method of claim 25, further comprising selecting an analyte binding ligand to which said analyte-analogue exhibits reversible binding.

31. The method of claim 25, wherein said detecting is selected from the group consisting of (a) measuring the appearance or disappearance of emission peaks, (b) measuring the ratio of the signal observed at two or more emission wavelengths, (c) measuring the appearance or disappearance of excitation peaks, (d) measuring the ratio of the signal observed at two or more excitation wavelengths, and combinations thereof.

32. The method of claim 25, wherein said detecting comprises measuring the change in the excited state lifetime of a first component of said non-radiative fluorescence resonance energy transfer donor-acceptor pair.

33. The method of claim 25, wherein said detecting comprises measuring the depolarization of fluorescence relative to excitation of a first component of said non-radiative fluorescence resonance energy transfer donor-acceptor pair.

34. A method of identifying an analyte-ligand binding pair that exhibits non-radiative fluorescence resonance energy transfer, said method comprising:
a) determining a constant region on a ligand at which to attach at least one component of a non-radiative fluorescence resonance energy transfer donor-acceptor pair;
b) obtaining a predetermined analyte binding ligand from a combinatorial library comprising ligands comprising said predetermined constant region, said analyte binding ligand having been predetermined by contacting the combinatorial library with a first analyte-analogue, and selecting an analyte binding ligand capable of binding the first analyte-analogue; and
c) attaching a label comprising at least one of a first component and a second component of said non-radiative fluorescence resonance energy transfer donor-acceptor pair to at least one of said analyte binding ligand and a second analyte-analogue such that non-radiative fluorescence resonance energy transfer occurs when said second analyte-analogue is bound to said analyte binding ligand, and a change in non-radiative fluorescence resonance energy transfer occurs when said second analyte-analogue is not bound to said analyte binding ligand.

35. The method of claim 34, comprising
attaching a label comprising said first component of said non-radiative fluorescence resonance energy transfer donor-acceptor pair to said analyte binding ligand at said predetermined constant region on said analyte binding ligand; and
attaching a label comprising said second component of said non-radiative fluorescence resonance energy transfer donor-acceptor pair to at least one of said analyte binding ligand and said second analyte-analogue.

36. The method of claim 34, further comprising:
preparing a combinatorial library comprising ligands comprising said constant region;
contacting said combinatorial library with a first analyte-analogue; and
identifying a ligand to which the first analyte-analogue binds, said ligand being the analyte binding ligand.

37. The method of claim 36, wherein said preparing comprises attaching a label comprising at least one component of said non-radiative fluorescence resonance energy transfer donor acceptor pair to said constant region of said ligands of said combinatorial library.

38. The method of claim 34, wherein said constant region of said ligands comprises at least one component of said non-radiative fluorescence resonance energy transfer donor acceptor pair.

39. The method of claim 34, wherein said second analyte-analogue comprises a predetermined constant region capable of binding at least one component of said non-radiative fluorescence resonance energy transfer donor-acceptor pair.

40. The method of claim 39, further comprising
attaching a label comprising said first component of said non-radiative fluorescence resonance energy transfer donor-acceptor pair to said constant region of said analyte binding ligand; and
attaching a label comprising said second component of said non-radiative fluorescence resonance energy transfer donor-acceptor pair to said constant region of said second analyte-analogue.

41. The method of claim 39, further comprising selecting an analyte binding ligand to which said second analyte-analogue exhibits reversible binding.

42. The method of claim 34, further comprising selecting an analyte binding ligand to which said second analyte-analogue exhibits reversible binding.

43. A method of identifying an analyte-ligand binding pair that exhibits non-radiative fluorescence resonance energy transfer, said method comprising:
a) determining a region on an analyte-analogue at which to attach a component of a non-radiative fluorescence resonance energy transfer donor-acceptor pair;
b) preparing an analyte-analogue comprising said predetermined region;
c) contacting a combinatorial library comprising ligands with said analyte-analogue;
d) identifying a ligand to which said analyte-analogue binds, said ligand being the analyte binding ligand; and
e) attaching a label comprising at least one of a first component and a second component of a non-radiative fluorescence resonance energy transfer donor-acceptor pair to at least one of said analyte binding ligand and said analyte-analogue such that non-radiative fluorescence resonance energy transfer occurs when said analyte-analogue is bound to said analyte binding ligand, and a change in non-radiative fluorescence resonance energy transfer when said analyte-analogue is not bound to said analyte binding ligand.

44. The method of claim 43, comprising attaching at least one component of said non-radiative fluorescence resonance energy transfer donor acceptor pair to said constant region of said analyte-analogue.

45. The method of claim 43, further comprising selecting an analyte binding ligand to which said analyte-analogue exhibits reversible binding.

46. The method of claim 43, wherein said identifying and said selecting occur simultaneously or substantially simultaneously.

47. A method of identifying an analyte-ligand binding pair that exhibits non-radiative fluorescence resonance energy transfer, said method comprising:
a) identifying a linking moiety to which at least one component of a non-radiative fluorescence resonance energy transfer donor-acceptor pair binds;
b) obtaining a predetermined analyte binding ligand from a combinatorial library comprising ligands, said analyte binding ligand having been predetermined by contacting the combinatorial library with a first analyte-analogue, and selecting an analyte binding ligand capable of binding the first analyte-analogue; and
c) attaching a label to said linking moiety, said label comprising a first component of a non-radiative fluorescence resonance energy transfer donor-acceptor pair;
d) attaching a label comprising a second component of said non-radiative fluorescence resonance energy transfer donor-acceptor pair to at least one of said analyte binding ligand and a second analyte-analogue; and
e) attaching said linking moiety to said analyte binding ligand,
wherein non-radiative fluorescence resonance energy transfer occurs when said second analyte-analogue is bound to said analyte binding ligand, and a change in non-radiative fluorescence resonance energy transfer when said second analyte-analogue is not bound to said analyte binding ligand.

48. The method of claim 47, comprising attaching said label to said linking moiety prior to attaching said moiety to said analyte binding ligand.

49. A method of screening a combinatorial library, said method comprising
a) preparing a combinatorial library comprising ligands comprising a first component of a non-radiative fluorescence resonance energy transfer donor-acceptor pair;
b) contacting said combinatorial library with an analyte-analogue comprising a second component of a non-radiative fluorescence resonance energy transfer donor-acceptor pair; and
c) identifying an analyte-ligand binding pair that exhibits non-radiative fluorescence resonance energy transfer.

50. A sensor comprising:
an analyte-ligand binding pair comprising
a) a first analyte-analogue, and
b) a predetermined analyte binding ligand, said analyte binding ligand having been predetermined by contacting a combinatorial library with a second analyte-analogue and selecting a ligand to which the second analyte-analogue binds,
c) a label comprising a first component and a second component of a non-radiative fluorescence resonance energy transfer donor-acceptor pair,
said analyte-ligand binding pair exhibiting non-radiative fluorescence resonance energy transfer when the first analyte-analogue is bound to said analyte binding ligand, and a change in non-radiative fluorescence resonance energy transfer when the first analyte-analogue is not bound to said analyte binding ligand.

51. The sensor of claim 50, wherein said analyte binding ligand and said analyte analogue are reversibly bound to each other.

52. The sensor of claim 50, wherein said sensor further comprises a matrix surrounding said analyte ligand binding pair.

53. The sensor of claim 50, wherein said sensor further comprises a semipermeable membrane surrounding said analyte ligand binding pair.

54. A kit comprising the sensor of claim 50.

55. A method of making a sensor, said method comprising:
a) selecting an analyte-analogue;
b) attaching a label comprising a first component of a non-radiative fluorescence resonance energy transfer donor-acceptor pair to an analyte-analogue;
c) selecting an analyte binding ligand from a combinatorial library, said analyte binding ligand being capable of binding with said analyte-analogue;
d) attaching a label comprising a second component of a non-radiative fluorescence resonance energy transfer donor-acceptor pair to said analyte binding ligand; and
e) encapsulating said labeled analyte binding ligand and said labeled analyte-analogue,
said sensor exhibiting either
non-radiative fluorescence resonance energy transfer when said analyte-analogue is bound to said analyte binding ligand, and a change in non-radiative fluorescence resonance energy transfer when said analyte-analogue is not bound to said analyte binding ligand, or
being free from non-radiative fluorescence resonance energy transfer when said analyte-analogue is bound to said analyte binding ligand, and exhibiting non-radiative fluorescence resonance energy transfer when said analyte-analogue is bound to said analyte binding ligand.
